(19) United States
(12) Patent Application Publication
(10) Pub. No.: US 2012/0209083 A1
(43) Pub. Date: Aug. 16, 2012

(54) METHOD OF LOCATING AN INGESTED CAPSULE

(75) Inventor: John R. Semler, Williamsville, NY (US)

(73) Assignee: THE SMART PILL CORPORATION, Buffalo, NY (US)

(21) Appl. No.: 13/326,045

(22) Filed: Dec. 14, 2011

Related U.S. Application Data
(63) Continuation of application No. 11/899,544, filed on Sep. 6, 2007, now abandoned.
(60) Provisional application No. 60/843,038, filed on Sep. 8, 2006.
(60) Provisional application No. 60/930,451, filed on May 16, 2007.

(51) Int. Cl. A61B 5/145; A61B 5/03

(52) U.S. Cl. 600/301

(57) ABSTRACT

A method of determining the location of an ingested capsule comprising the steps of providing an ingestible capsule having a pH sensor and a pressure sensor, having a subject ingest the capsule, recording pH measurements from the pH sensor as a function of time as the capsule moves through at least a portion of the gastrointestinal tract of the subject, recording pressure measurements from the pressure sensor as a function of time as the capsule moves through at least a portion of the gastrointestinal tract of the subject, deriving a frequency of contractions pressure pattern as a function of time and the pressure measurements, and identifying an appreciable variation in the frequency pressure pattern in substantially the same time period as an appreciable variation in the pH to determine the capsule's location at a first position in the gastrointestinal tract of the subject.

### CROSS-REFERENCE TO RELATED APPLICATIONS

This application is a continuation of U.S. patent application Ser. No. 11/899,544, filed Sep. 6, 2007, which claims the benefit of U.S. Provisional Patent Application No. 60/843,038, filed Sep. 8, 2006, and U.S. Provisional Patent Application No. 60/930,451, filed May 16, 2007. The entire content of these applications are incorporated by reference herein.

### TECHNICAL FIELD

The present invention relates generally to ingestible capsules and, more particularly, to a process for determining the location of an ingested capsule as it transitions between segments of the digestive tract.

### BACKGROUND ART

Ingestible capsules are well-known in the prior art. Such capsules are generally small pill-like devices that can be ingested or swallowed by a patient. It is known that such capsules may include one or more sensors for determining physiological parameters of the gastrointestinal tract, such as sensors for detecting temperature, pH, pressure and the like.

A number of methods of determining location of an ingestible capsule are known in the prior art. For example, it is known that signal strength or signal triangulation may be used to attempt to determine the location of an ingested capsule. However, the use of an RF signal has a number of disadvantages, including that it generally requires multiple antennas, various tissues may impact the signal differently, and patient movement may skew the results. It is also known that accelerometers may be used to attempt to determine location, but such methods also have disadvantages, such as drift, non-linear progression and rotational inaccuracy.

It is also known that certain physiological parameters may be associated with regions of the gastrointestinal tract. For example, a 1988 article entitled “Measurement of Gastrointestinal pH Profiles in Normal Ambulant Human Subjects” discloses pH measurements recorded by a capsule passing through the gastrointestinal tract. While pH has been correlated with transitions from the stomach to the small bowel (gastric emptying) and from the distal small bowel to the colon (ileo-caecal transition), often there are not significant pH variations correlated with certain regions of the gastrointestinal tract, and patients with gastrointestinal maladies may have abnormal readings.

Thus, there is need for accurately determining when an ingestible capsule moves from one segment of the gastrointestinal tract to another.

### BRIEF SUMMARY OF THE INVENTION

With parenthetical reference to corresponding parts, portions or surfaces of the disclosed embodiment, merely for the purposes of illustration and not by way of limitation, the present invention provides an improved method for determining the movement of an ingestible capsule from a first segment of the gastrointestinal tract to a second segment of the gastrointestinal tract comprising the steps of providing an ingestible capsule (20) having a pH sensor (22) and a pressure sensor (23), ingesting the capsule, recording pressure measurements and pH measurements from the ingestible capsule as it moves through the gastrointestinal tract, deriving a pressure pattern as a function of time and the pressure measurements, monitoring for a variation in pH, and determining if there is an appreciable variation in the pressure pattern at such time period, whereby the capsule's location at a first position may be determined. The step of deriving a pressure pattern as a function of time and the pressure measurements may comprise the step of conditioning the recorded pressure measurements. The conditioning may comprise the step of normalizing the pressure measurements by applying a baseline compensation, and the baseline may be about 3 mmHg. The conditioning may comprise the steps of filtering out data points in the pressure measurements above an upper limit and filtering out data points in the pressure measurements below a lower limit, and the upper limit may be about 200 mmHg and the lower limit may be about 9 mmHg. The method may comprise the step of comparing such pH variation and such variation in frequency of contractions to a reference template. The method may comprise determining if there is an appreciable variation in motility index at such time period. The method may further comprise the steps of monitoring for a second variation in pH and determining if there is an appreciable variation in the frequency of contractions at such second time period, whereby the capsule's location at a second position may be determined. The method may comprise the step of determining transit time between the first position and the second position.

Accordingly, the general object is to provide a method for determining the movement of an ingestible capsule from a first segment of the gastrointestinal tract to a second segment of the gastrointestinal tract based on pressure and pH.

Another object is to provide a method for confirming the movement of a capsule from a first segment of the gastrointestinal tract to a second segment of the gastrointestinal tract based on pressure patterns.

Another object is to provide a method for determining the movement of an ingestible capsule from a first segment of the gastrointestinal tract to a second segment of the gastrointestinal tract based on frequency of contractions.

Another object is to provide a method for determining the movement of an ingestible capsule from a first segment of the gastrointestinal tract to a second segment of the gastrointestinal tract as a function of the area under a curve of pressure readings versus time.

Another object is to provide a method for determining the movement of an ingestible capsule from a first segment of the gastrointestinal tract to a second segment of the gastrointestinal tract as a function of the amplitude and/or frequency of pressure readings.

Another object is to provide a method for determining transit time of a capsule through one or more segments of the gastrointestinal tract.

These and other objects and advantages will become apparent from the foregoing and ongoing written specification, the drawings, and the claims.

### BRIEF DESCRIPTION OF THE DRAWINGS

FIG. 1 is a prior art graphical view of pH readings taken by a radio telemetry capsule passing through the gastrointestinal tract. FIG. 1 also shows various segments of the gastrointestinal tract.

FIG. 2 is a graph of pH versus time taken by a capsule passing through the gastrointestinal tract.

FIG. 3 is a graph of pressure over the same period of time shown in FIG. 2 taken by the capsule.

FIG. 4 is a graph of the number of contractions during five minute intervals over the same period of time shown in FIG. 2.

FIG. 5 is a graph of the normalized relative motility index for five minute intervals over the same period of time shown in FIG. 2.

FIG. 6 is a graph of pH, pressure and motility centered around passage of the capsule through the ileo-caecal junction.

FIG. 7 is a sectional view of an ingestible capsule for providing pressure and pH data in FIGS. 2-3.

### DETAILED DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly', the terms “inwardly” and “outwardly” generally refer to the orientation of a surface relative to its axis of elongation, or axis of rotation, as appropriate.

A method is provided for determining the movement of an ingestible capsule from a first segment of the gastrointestinal tract to a second segment of the gastrointestinal tract as a function of pressure readings and pH readings taken by the ingested capsule.

A capsule 20 is ingested by a subject and readings from sensors on the capsule are taken as the capsule passes through the gastrointestinal tract of the subject. Data from the pressure sensor and pH sensor are collected and analyzed by comparison to a reference template and/or to each other, to determine the location of the capsule. In a first embodiment, variations in pH and motility index patterns are used to mark the transition of the ingested capsule from the distal ileum to the caecum. Pressure patterns are used to confirm whether or not a variation in pH, as compared to a reference template, denotes a transition from the distal small bowel or distal ileum to the right colon or caecum. Thus, pH and pressure patterns are compared to reference data and used to determine an ingested capsule's passage through the ileo-caecal junction. In a second embodiment, variations in pH and motility index patterns are used to mark the transition of the ingested capsule from the stomach to the small bowel.

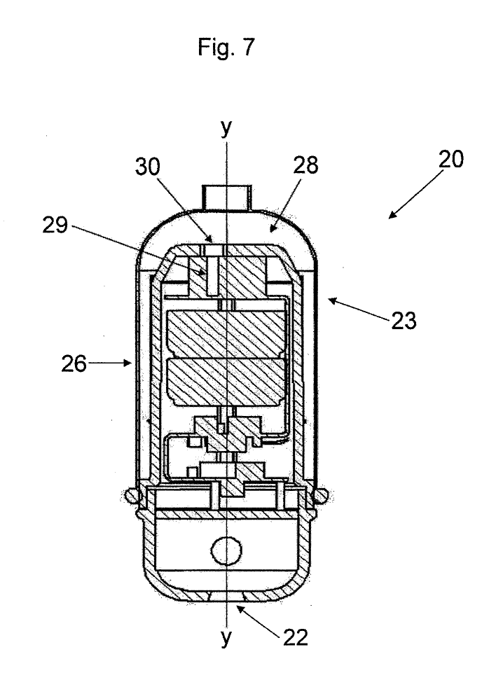

As shown in FIG. 7, capsule 20 is an elongated ellipsoid-shaped device, somewhat resembling a medicament capsule. The capsule generally has a hard shell or casing which houses the transmitting electronics, battery compartment and sensors. Capsule 20 is adapted to be ingested or otherwise positioned within a tract to sense both pressure and pH within the tract and to transmit such readings. As shown, capsule 20 is generally a cylindrical member elongated about axis y-y and having generally rounded closed ends. The capsule is generally provided with an outer surface to facilitate easy swallowing of the capsule.

Capsule 20 includes a pressure sensor assembly 23 comprising a flexible sleeve 26 affixed to the shell of the capsule and defining a chamber 28 between the shell and the sleeve. A pressure sensor 29 is operatively arranged to sense pressure within chamber 28 and communicates with the chamber through a fluid port 30 at one end of the shell of the capsule. As shown, the pressure sleeve 26 of capsule 20 extends from a point below the middle of the capsule up over the top end of the capsule.

On the opposite end of capsule 20 to pressure sensor 23 is pH sensor 22. In the preferred embodiment, pH sensor 22 is a conventional ISFET type pH sensor. ISFET stands for ion-selective field effect transistor and the sensor is derived from MOSFET technology (metal oxide screen field effect transistor). A current between a source and a drain is controlled by a gate voltage. The gate is composed of a special chemical layer which is sensitive to free hydrogen ions (pH). Versions of this layer have been developed using aluminum oxide, silicon nitride and titanium oxide. Free hydrogen ions influence the voltage between the gate and the source. The effect on the drain current is based solely on electrostatic effects, so the hydrogen ions do not need to migrate through the pH sensitive layer. This allows equilibrium, and thus pH measurement, to be achieved in a matter of seconds. The sensor is an entirely solid state sensor, unlike glass bulb sensors which require a bulb filled with buffer solution. Only the gate surface is exposed to the sample.

In the preferred embodiment, the capsule transmits sensed data at about 434 MHz and measures 26.8 mm long by 11.7 mm in diameter. A portable data receiver worn by the subject receives and stores data transmitted by the capsule. Software performs data analysis and presents a graphical data display of pH, pressure and temperature readings for analysis. After activation and ingestion, the capsule senses and transmits data for at least 120 hours after activation. The pH, pressure and temperature data are transmitted from within the GI tract to the data receiver. In the preferred embodiment, the range and accuracy of the sensors are generally 0.05 to 9.0 pH units with an accuracy of ±0.5 pH units, 0 to 350 mmHg with an accuracy of 5 mmHg, or 10% above 100 mmHg, and 25° to 49° C. with an accuracy of ±1° C. The data receiver contains rechargeable batteries and when seated in a docking station allows for battery charging and data download. Data is downloaded from the data receiver through the docking station via USB connection to a Windows PC compatible laptop.

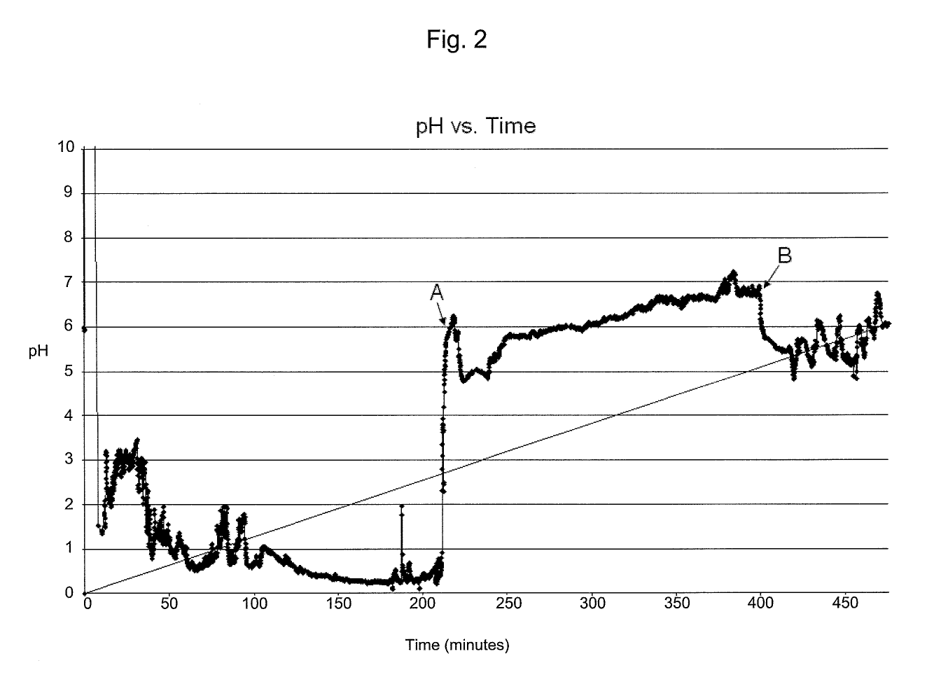

The pH readings from the ingested capsule are plotted against time, as shown in FIG. 2. Based on reference data, a substantial variation or increase in pH, generally indicated at A, indicates passage of the capsule from the stomach to the small intestine, often referred to as gastric emptying. A latter variation in pH, indicated at B, suggests movement of the capsule from the ileum to the caecum. It has been found that this significant pH drop is seen some hours after gastric emptying and is due to the capsule moving from the ileum to the caecum, a transition referred to as the ileo-caecal junction.

However, not only is a variation in pH patterns used to determine that the capsule is at the junction between the stomach and small bowel or at the ileo-caecal junction, but an associated change in pressure pattern is also employed. In the preferred embodiment, pressure patterns derived from pressure measurements taken by the capsule as it passes through the gastrointestinal tract are used. In the preferred embodiment, the pressure data from the subject is conditioned to distinguish real contraction data from artifacts or “noise” within the data set, as well as to discount physiologically improbable values. In the preferred embodiment, both concerns are addressed as part of a process which inspects each data value in the pressure measurement data set provided by the capsule. Because the conditioning utilizes constant minimum and maximum threshold values to determine and eliminate data spikes and artifacts, the input pressure data is baseline compensated. As mentioned above, the pressure data is then conditioned by filtering out those sets of data points or contractions whose peaks are above a predetermined threshold or limit. In the preferred embodiment, this threshold is about 200 mmHg. In addition, those contraction patterns whose peaks are less than a predetermined threshold or limit are also filtered out. In the preferred embodiment, this minimum threshold is about 9 mmHg. Thus, in the preferred embodiment the process considers a set of baseline compensated pressure measurements and begins evaluating each value in linear sequence from beginning to end. If a point is found to exceed the defined maximum, then the high value or spike is removed with its associated ascending and descending artifact values by traversing the data set both behind and ahead of the detected spike and zeroing the spike and any associated values, until either its termination or a new contraction is detected. The determination that an artifact has terminated is defined as any data point below a minimum pressure value. Contrarily, finding the next contraction from the high value is based on the detection of three consecutive ascending values, which is interpreted as an ascent in pressure, indicating the edge of a different contraction. Thus, in determining, for example, the area under the curve for a given time interval, a pressure point is included in the calculation only if its value is greater than or equal to the sum of the baseline pressure and the minimum threshold and is below the sum of the baseline pressure and the maximum threshold.

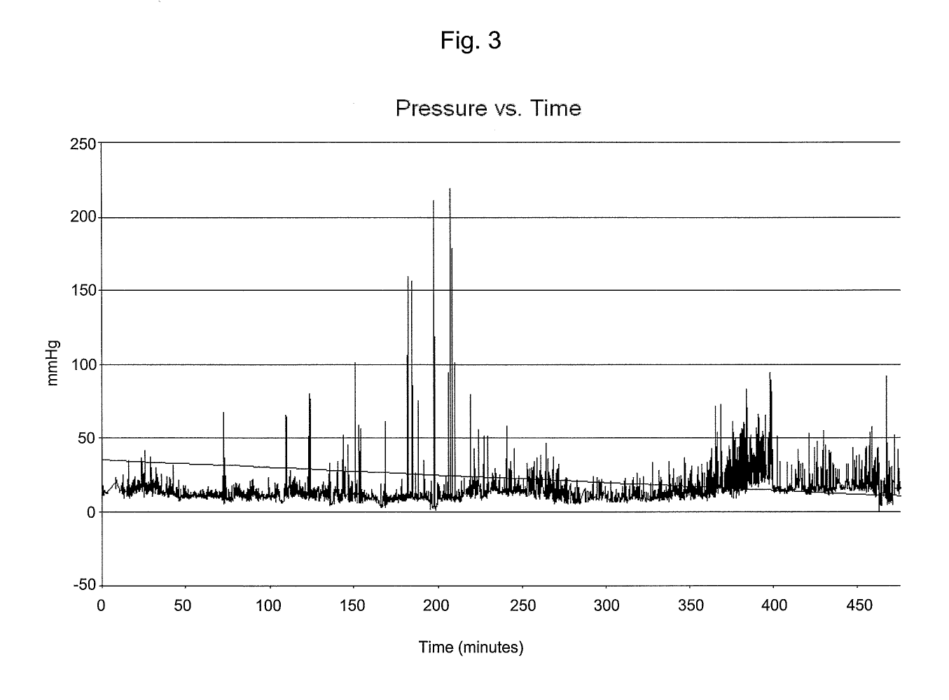

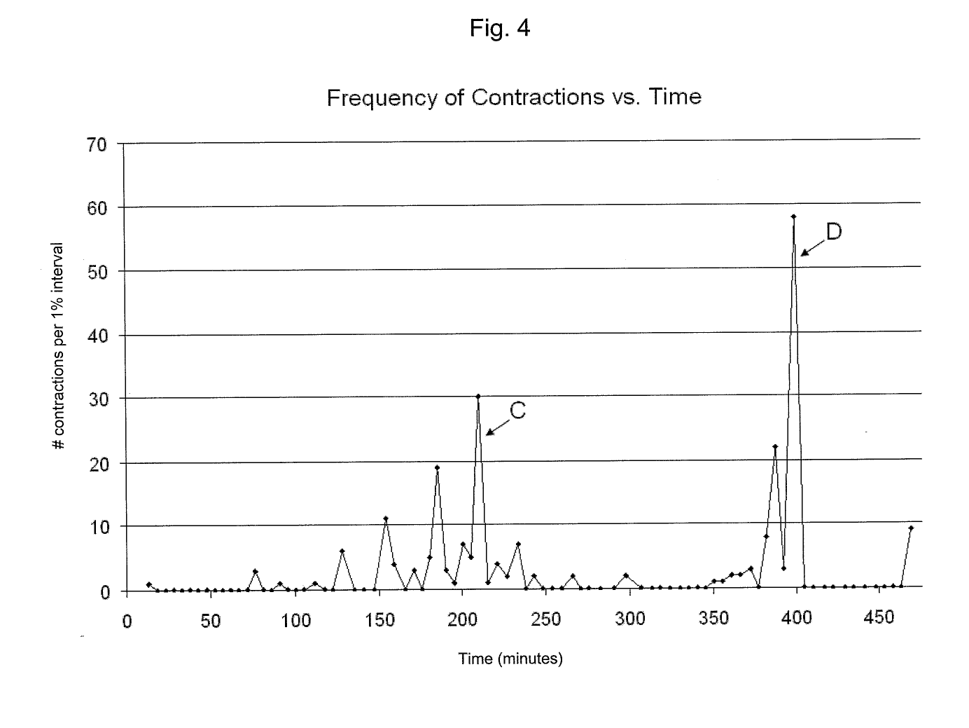

Average pressure readings from the capsule plotted against transit time are shown in FIG. 3. The number of contractions over a baseline for a given time interval, five minutes in the preferred embodiment, plotted against the same overall time period are shown in FIG. 4. In the preferred embodiment, a contraction is designated by an increase in pressure over 10 mmHg and the subsequent return below 10 mmHg. However, it is contemplated that gastrointestinal contractions may be determined based on other variations in pressure or baselines other than 10 mmHg.

As shown in FIG. 4, a variation in the frequency of contractions was generally found to occur, as indicated at C, at a time corresponding to the gastric emptying suggested by the graph of pH shown in FIG. 2. This correlation between the variation in frequency of contractions C and the variation in pH A is used as a reference to confirm that the capsule has moved from the stomach to the small bowel. A further and more substantial variation in contractions occurs, as indicated at D, at a time corresponding to the ileo-caecal junction suggested by the graph of pH shown in FIG. 2. This correlation between the variation in frequency of contractions D and the variation in pH B is used as a reference to determine that the capsule has moved from the ileum to the caecum of the subject.

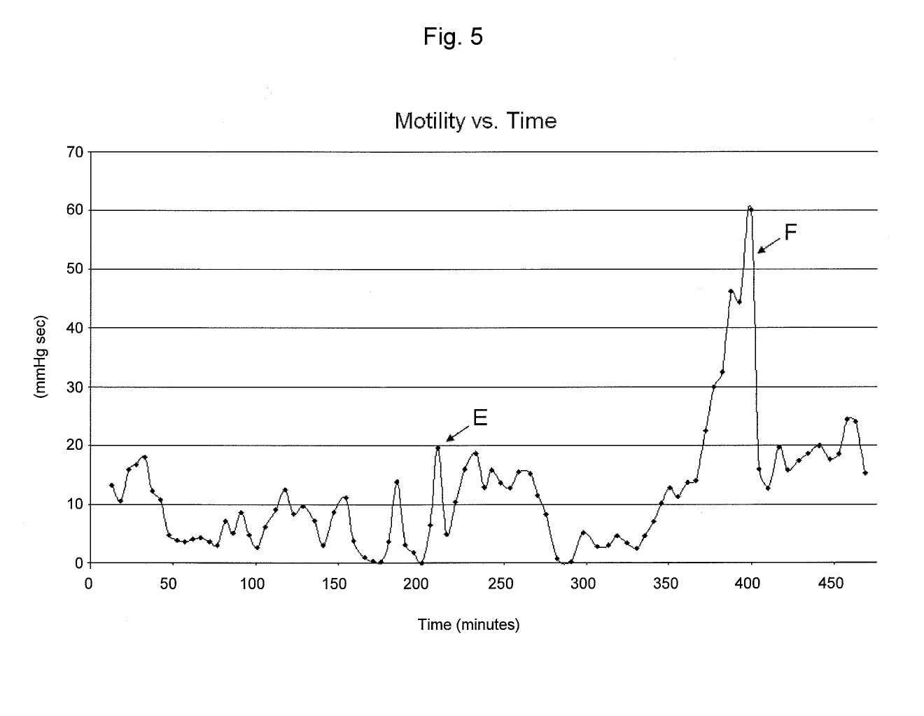

FIG. 5 is a plot of the normalized relative motility index at five minute intervals versus time. Each data point is the area under the curve of the graph of pressure shown in FIG. 3 for five minute intervals. Motility index as used herein is the area under the curve (or the integral of pressure over a time region) divided by the size of the time region. While a five minute time region is used in this graph, other time periods may be employed. Plotted against transit time, generally a substantial variation occurs, indicated at F, at substantially the same time as the variation B in pH. This variation in motility index is used in the preferred embodiment as a reference to confirm that the capsule has moved from the ileum to the caecum of the subject. Also, a variation in motility index indicated at E may be used as a reference with pH variation A to confirm that the capsule has moved from the stomach to the small intestine.

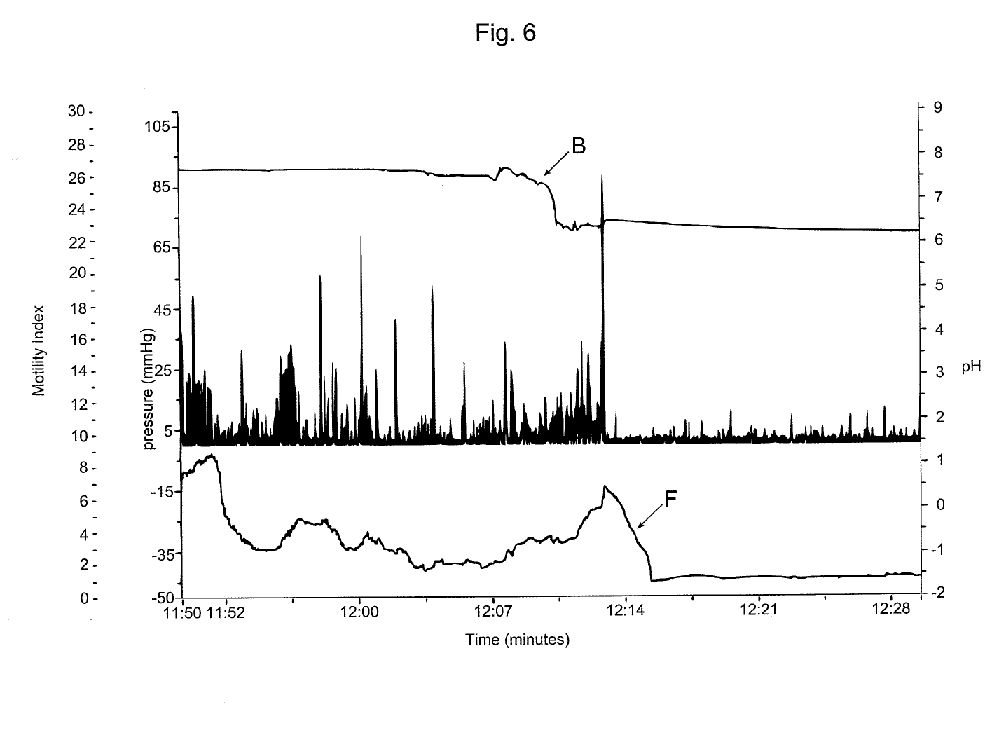

FIG. 6 is a representative graph of pH and conditioned pressure readings for a subject, together with motility index, for the twenty minutes prior to passing through the ileo-caecal junction and twenty minutes after passing through the ileo-caecal junction. As shown, the motility index stabilizes and flattens out after passage through the ileo-caecal junction.

By basing location on both pH and pressure patterns, one can more accurately determine the movement of ingested capsule 20 from one segment of the gastrointestinal tract to a second segment of the gastrointestinal tract of a subject. In comparing patterns from a subject with the reference templates for both pH and pressure, if there is a correlation between a variation in pH B and a variation in frequency of contractions D and/or motility index F, then a determination of the capsule's location may be more accurate. Without this correlation, the capsule being located at or near the ileo-caecal junction is less certain.

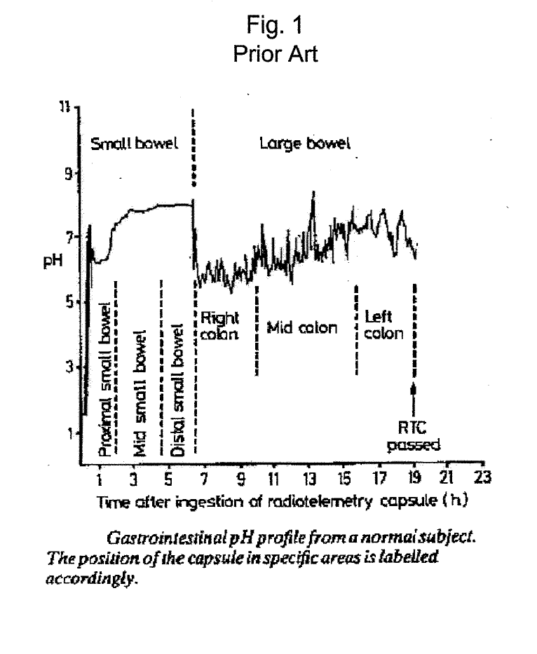

The patterns indicate that the intraluminal environment of the gastrointestinal tract as it transitions from the small intestine into the colon changes. The caecum, as compared to the distal ileum, is a less contractile reservoir where colonic bacteria cause an acidic change in pH. Thus, in the preferred embodiment, capsule 20 is ingested by the subject and pH readings and pressure readings are taken and compared as indicated above. Certain pH reference values are known in the prior art, as shown in FIG. 1. In addition, reference patterns, from which reference templates FIG. 2-5 were derived, were formed from capsule testing data. One hundred and four volunteers swallowed an ingestible capsule having a pH sensor and a pressure sensor after an overnight fast, together with a standardized meal and 100 cc's of water. It was found that a rapid pH change from acidic to alkaline (greater than 4 and at least a 3 unit rise from baseline gastric pH) marked the emptying of the ingested capsule from the stomach into the duodenum or small bowel. On the capsule's recordings, approximately 5.5 hours after the capsule's gastric emptying, a drop in pH of greater than 1 unit for more than 5 minutes was generally found. The frequency and the amplitude of contractions were analyzed from 30 minutes before the beginning of the pH drop to 30 minutes after. These parameters were then compared by two-sample unequal variance t test. The results of the test showed that average time from the gastric emptying to the pH drop was 5 hours and 23 minutes. The frequency of contractions for the 30 minutes before the pH drop was shown to be 3.9 contractions per minute (95% CI 3.99±0.014), and for the 30 minutes after the drop was 2.1 contractions per minute (95% CI 2.1±0.01), p<0.0001. The mean amplitude of contractions was no different between the time periods chosen (19.6 mmHg before, 19.4 mmHg after the pH drop, p=0.8). The motility index for the 30 minutes before the pH change was 1.54 and the motility index for the 30 minutes after the pH change was 0.91, p<0.0001.

Readings from a subject may be compared to the reference templates to determine location. Thus, a change in pH and a change in either frequency of contractions or motility index that correlates with the variations in the template may be used to determine location. In the preferred embodiment, the combined change in pH and motility index is used to mark the transition between the distal ileum and the caecum. By using patterns based on both pH and pressure, location is more accurate because changes in pH based on bacterial overgrowth or malignancies in the gastrointestinal tract are not assumed to be a transition from one segment to a second segment if they are not accompanied by a corresponding variation in the frequency of contractions or motility index.

With the determination that the capsule has passed from the stomach to the small bowel and then through the ileo-caecal junction, transit time through the small bowel is ascertained. Transit time through the colon can then be determined as well. This is useful in a number of clinical applications.

The present invention contemplates that many changes and modifications may be made. Therefore, while the presently-preferred form of the improved method has been shown and described, and a number of alternatives discussed, persons skilled in this art will readily appreciate that various additional changes and modifications may be made without departing from the spirit of the invention, as defined and differentiated by the following claims.

1. A method of determining movement of an ingested capsule from a first segment of a gastrointestinal tract to a second segment of said gastrointestinal tract comprising the steps of:
providing an ingestible capsule having a pH sensor and a pressure sensor;
having a subject ingest said capsule;
recording pH measurements from said pH sensor as a function of time as said capsule moves through at least a portion of said gastrointestinal tract of said subject;
recording pressure measurements from said pressure sensor as a function of time as said capsule moves through at least a portion of said gastrointestinal tract of said subject;
deriving a frequency of contraction pressure pattern as a function of time and said pressure measurements; and
identifying an appreciable variation in said frequency pressure pattern in substantially the same time period as an appreciable variation in said pH to determine said capsule's location at a first position between the ileum and the caecum of said gastrointestinal tract of said subject.

2. The method set forth in claim 1, wherein said step of identifying an appreciable variation in said frequency pressure pattern in substantially the same time period as an appreciable variation in said measured pH comprises the steps of:
providing a reference pH and a reference frequency pressure pattern; and
analyzing said pH variations for said subject and said frequency pressure pattern variations for said subject relative to said respective references.

3. The method set forth in claim 1, wherein said frequency pressure pattern comprises frequency of contractions relative to a baseline over a given time interval.

4. The method set forth in claim 1, wherein said appreciable variation in said frequency pressure pattern is identified by an algorithm.

5. The method set forth in claim 1, and further comprising the step of identifying a second appreciable variation in said frequency pressure pattern in substantially the same time period as a second appreciable variation in said measured pH to determine said capsule's location at a second position between the stomach and the small bowel of said gastrointestinal tract of said subject.

6. The method set forth in claim 5, and further comprising the step of determining transit time between said first position and said second position.

7. A method of determining movement of an ingested capsule from a first segment of a gastrointestinal tract to a second segment of said gastrointestinal tract comprising the steps of:
providing an ingestible capsule having a pH sensor and a pressure sensor;
having a subject ingest said capsule;
recording pH measurements from said pH sensor as a function of time as said capsule moves through at least a portion of the gastrointestinal tract of said subject;
recording pressure measurements from said pressure sensor as a function of time as said capsule moves through at least a portion of said gastrointestinal tract of said subject;
deriving a frequency of contraction pressure pattern as a function of time and said pressure measurements;
identifying an appreciable variation in said pH and frequency pressure pattern to determine said capsule's location at a first position; and
identifying an appreciable variation in said pH and frequency pressure pattern to determine said capsule's location at a second position.

8. The method set forth in claim 7, wherein said step of identifying an appreciable variation in said frequency pressure pattern comprises the steps of:
providing a reference frequency pressure pattern; and
analyzing said frequency pressure pattern variations for said subject relative to said reference.

9. The method set forth in claim 7, wherein said step of identifying an appreciable variation in said pH comprises the steps of:
providing a reference pH; and
analyzing said pH variations for said subject relative to said reference.

10. The method set forth in claim 9, wherein said reference pH is a sustained pH change that exceeds about 3.

11. The method set forth in claim 7, wherein said frequency pressure pattern comprises frequency of contractions relative to a baseline over a given time interval.

12. The method set forth in claim 7, wherein said appreciable variation in said frequency pressure pattern and said appreciable variation in said pH is identified by an algorithm.

13. The method set forth in claim 7, and further comprising the step of determining transit time between said first position and said second position.

14. The method set forth in claim 7, wherein said first location is a junction between the stomach and the small bowel of said gastrointestinal tract of said subject and said second location is a junction between the ileum and the caecum of said gastrointestinal tract of said subject.

15. A method of determining movement of an ingested capsule from a first segment of a gastrointestinal tract to a second segment of said gastrointestinal tract comprising the steps of:
providing an ingestible capsule having a pH sensor and a pressure sensor;
having a subject ingest said capsule;
recording pH measurements from said pH sensor as a function of time as said capsule moves through at least a portion of said gastrointestinal tract of said subject;
recording pressure measurements from said pressure sensor as a function of time as said capsule moves through at least a portion of said gastrointestinal tract of said subject;
deriving a frequency of contractions pressure pattern as a function of time and said pressure measurements;
providing a reference pH and a reference frequency pressure pattern;
analyzing said pH variations for said subject and said frequency pressure pattern variations for said subject relative to said respective references to determine said capsule's location at a first position.

16. The method set forth in claim 15, wherein said frequency pressure pattern comprises frequency of contractions relative to a baseline over a given time interval.

17. The method set forth in claim 15, and further comprising the steps of analyzing said pH variations for said subject and said pressure pattern variations for said subject relative to said respective references to determine said capsule's location at a second position.

18. The method set forth in claim 17, and further comprising the step of determining transit time between said first position and said second position.

19. The method set forth in claim 17, wherein said first location is a junction between the stomach and the small bowel of said gastrointestinal tract of said subject and said second location is a junction between the ileum and the caecum of said gastrointestinal tract of said subject.
